(19) United States
(12) Patent Application Publication
(10) Pub. No.: US 2026/0216052 A1
(43) Pub. Date: Jul. 30, 2026

(54) COSMETIC COMPOSITION FOR ENHANCING RESISTANCE TO AGING

(71) Applicant: L'OREAL, Paris (FR)

(72) Inventors: Stephen Matthew LYNCH, Westfield, NJ (US); Xi Yan, Berkeley Heights, NJ (US); Zhiming Zhang, Shanghai (CN)

(73) Assignee: L'OREAL, Paris (FR)

(21) Appl. No.: 19/041,428

(22) Filed: Jan. 30, 2025

(51) Int. Cl. A61K 8/9789; A61K 8/60; A61Q 19/08

(52) U.S. Cl. CPC A61K 8/9789; A61K 8/602; A61Q 19/08; A61K 2800/5922; A61K 2800/782

(57) ABSTRACT

A cosmetic composition includes blueberry extract, pomegranate extract, dipotassium glycyrrhizate, and hydroxypropyl tetrahydropyrantriol in a cosmetic carrier system, wherein the cosmetic composition demonstrates the unexpected effect of a significant reduction of expression of several genes known to be involved in the skin aging process, for example a reduction of expression in a range from about 40% for marker gene MAP3K5 and about 50% for each of marker genes IFT57 and MFHAS1. The cosmetic carrier system that may be an emulsion, for example a silicone emulsion having a thick cream texture, or other type of emulsion, a gel, a single phase, or a bi-phase, and wherein the cosmetic composition is in the form of a gel-cream, cream, gel, lotion, toner, or serum, and may be provided and intended to leave on, rinse off, or use as a cleanser.

### FIELD OF THE INVENTION

The present disclosure relates to a water-based cosmetic composition to combat skin aging that includes a novel blend of skin actives which demonstrate reduction in expression of certain proteins associated with adverse cell processes and aging.

### BACKGROUND OF THE INVENTION

Skin aging and the attendant visible and textural changes to skin are a major focus of a wide variety of cosmetic products. Skin on the face and neck are particularly important targets for such treatments, especially skin on the neck which has different physical characteristics than skin on the face, and consequently shows aging in particular ways, which can include lines, wrinkles, crepiness, and sagging. Among skin cleansers, toners, moisturizers and other forms of treatment, a wide variety of skin care products provide blends of ingredients that purport to address the signs of aging.

While many ingredients in these products are known to have relevant biological effects, it remains a challenge to provide cosmetic treatments that include amounts and combinations of such ingredients sufficient to be effective.

One aspect of skin aging is understood to be affected by the expression of certain genes which produce proteins that have been studied and identified as affecting processes such as stress response, apoptosis and inflammatory response in cells. These negative processes are associated with expression of peptides by certain genes that can confer adverse effects on skin which manifest as signs of aging. Another aspect of skin aging is the accumulation of advanced glycation end-products (sometimes referred to in the cosmetics literature as “AGEs”) in skin, which is implicated in the process of skin aging related to collagen degradation (correlated with loss of skin firmness) and melanogenesis (correlated with loss of skin tone evenness). Active ingredients with anti-glycation activity are known, including, for example, aminoguanidine (also known as gallic acid, extracted from hazelnut skins), alisin (extracted from garlic), and carnosine (also known as beta-alanyl-L-histidine, a dipeptide found in mammals), and may be found in some existing skin care products.

The present invention provides a novel combination of active ingredients showing a surprising statistically significant reduction in expression of certain proteins associated with adverse cell processes and aging, wherein one or more of the ingredients alone and in sub-combinations, as compared with the novel combination, do not demonstrate a similar effect on cells.

### BRIEF SUMMARY OF THE INVENTION

In an embodiment, the disclosure provides a cosmetic composition comprising a novel blend of skin actives comprising blueberry extract, pomegranate extract, dipotassium glycyrrhizate, and hydroxypropyl tetrahydropyrantriol in a cosmetic carrier system, wherein the cosmetic composition demonstrates the unexpected effect of a significant reduction of expression of several proteins known to be involved in the skin aging process, for example a reduction of expression of the proteins in a range from about 40% for marker gene MAP3K5 and about 50% for each of marker genes IFT57 and MFHAS1. The skin actives are provided in a cosmetic carrier system that may be an emulsion, for example a silicone emulsion or reverse emulsion having thick creamy texture with medium playtime, and velvety skin finish, that is easy to apply and absorb, as exemplified herein. The novel blend of skin actives may also be provided in other cosmetic formats such as other types of emulsion, gel, gel-cream, single phase, or bi-phase, and wherein the cosmetic composition is in the form of one of a thick cream, gel-cream, gel, lotion, toner, or serum, and may be provided and intended to leave on, rinse off, or use as a cleanser or spot treatment.

In an embodiment, the disclosure provides a cosmetic composition comprising: a blend of skin actives comprising blueberry extract; pomegranate extract; dipotassium glycyrrhizate; and hydroxypropyl tetrahydropyrantriol; and a cosmetic carrier system, wherein the cosmetic composition confers a statistically significant reduction in the protein expression of at least one gene selected from the group consisting of mitogen-activated protein kinase 5 [MAP3K5], Intraflagellar transport protein 57 [IFT57], Multifunctional ROCO Family Signaling Regulator 1 [MFHAS1], and combinations thereof.

In some embodiments, the cosmetic carrier system comprises or more ingredients selected from the group consisting of water, oils, hydrating agents, thickeners, emulsifiers, surfactants, water based solvents, oil based solvents, additional skin actives, fillers, preservatives, chelating agents, pH adjusters, dispersants, pigments, effect pigments, dyes, colorants, powders, opacifiers, fragrances, and combinations thereof. In some embodiments, the cosmetic composition is an emulsion, a gel, a single phase, or a bi-phase. In some embodiments, the cosmetic composition is in the form of a gel-cream, cream, gel, lotion, toner, or serum, and is intended to leave on, rinse off, or use as a cleanser. In some embodiments, blueberry extract in the form of Vaccinium myrtillus fruit extract is present in a range in a range from about 0.01% to about 5%, by weight, based on the weight of the composition. In some embodiments, pomegranate extract in the form of Punica granatum fruit extract is present in a range in a range from about 0.01% to about 5%, by weight, based on the weight of the composition. In some embodiments, dipotassium glycyrrhizate is present in a range in a range from about 0.05% to about 5%, by weight, based on the weight of the composition. In some embodiments, hydroxypropyl tetrahydropyrantriol is present in a range in a range from about 0.5% to about 15%, by weight, based on the weight of the composition. In some embodiments, blueberry extract in the form of Vaccinium myrtillus fruit extract is present at about 0.08%, pomegranate extract in the form of Punica granatum fruit extract is present at about 0.02%, dipotassium glycyrrhizate is present at about 0.20%, and hydroxypropyl tetrahydropyrantriol is present at about 10.5%, all amounts by weight, based on the weight of the composition.

In an embodiment, the disclosure provides a blend of skin actives comprising blueberry extract; pomegranate extract; dipotassium glycyrrhizate; and hydroxypropyl tetrahydropyrantriol; and a cosmetic carrier system comprising: water; at least one silicone-based emulsifier; at least one silicone oil; at least one branched, liquid alkane; and at least one hydrating agent, wherein the cosmetic composition confers a statistically significant reduction in the protein expression of at least one gene selected from the group consisting of mitogen-activated protein kinase 5 [MAP3K5], Intraflagellar transport protein 57 [IFT57], Multifunctional ROCO Family Signaling Regulator 1 [MFHAS1], and combinations thereof. The composition may include additional emulsifiers, surfactants, and waxes, and other ingredients. In some embodiments, the cosmetic carrier system comprises or more ingredients selected from the group consisting of additional skin actives, fillers, preservatives, chelating agents, pH adjusters, dispersants, pigments, effect pigments, dyes, colorants, powders, opacifiers, fragrances, and combinations thereof. In some embodiments, in the cosmetic carrier system: the at least one silicone-based emulsifier is selected from the group consisting of dimethicone/PEG-10/15 crosspolymer, lauryl PEG-9 polydimethylsiloxyethyl dimethicone, dimethicone/polyglycerin-3 crosspolymer, cetyl PEG/PPG-10/1 dimethicone, PEG-18/PPG-18 dimethicone, PEG-10 dimethicone, PEG/PPG-18/18 methicone, lauryl PEG/PPG-18/18 methicone, PEG-9 polydimethylsiloxyethyl dimethicone, and combinations thereof; the at least one silicone oil is dimethicone; the at least one branched, liquid alkane is selected from the group consisting of isohexadecane, isododecane, isodecane, C15-19 alkane, and combinations thereof; the at least one hydrating agent is selected from the group consisting of glycerin, propylene glycol, dipropylene glycol, ethylhexyl hydroxystearate, synthetic wax, and combinations thereof; and comprises at least one additive selected from the group consisting of aluminum starch octenylsuccinate, bismuth oxychloride, cellulose, silica silylate, phenoxyethanol, sorbitol, sodium citrate, magnesium sulfate, and trisodium ethylenediamine disuccinate. In some embodiments, in the cosmetic carrier system: the water is present in a range from about 30% to about 40%; the at least one silicone-based emulsifier comprises dimethicone/PEG-10/15 crosspolymer, alone or in combination with lauryl PEG-9 polydimethylsiloxyethyl dimethicone, the dimethicone/PEG-10/15 crosspolymer present in a range in a range from about 3% to about 4%, the lauryl PEG-9 polydimethylsiloxyethyl dimethicone present in a range in a range from about 1% to about 1.5%; the at least one silicone oil comprises dimethicone present in a range in a range from about 5% to about 25%; the at least one branched, liquid alkane comprises isohexadecane present in a range from about 1% to about 15%; the at least one hydrating agent comprises glycerin, propylene glycol, ethylhexyl hydroxystearate, and synthetic wax; and at least one additive comprises aluminum starch octenylsuccinate, bismuth oxychloride, cellulose, silica silylate, phenoxyethanol, sorbitol, sodium citrate, magnesium sulfate, and trisodium ethylenediamine disuccinate, all amounts by weight, based on the weight of the composition. In some embodiments, blueberry extract in the form of Vaccinium myrtillus fruit extract is present in a range in a range from about 0.01% to about 5%, pomegranate extract in the form of Punica granatum fruit extract is present in a range in a range from about 0.01% to about 5%, dipotassium glycyrrhizate is present in a range in a range from about 0.05% to about 5%, and hydroxypropyl tetrahydropyrantriol is present in a range in a range from about 5% to about 15%, all amounts by weight, based on the weight of the composition. In some embodiments, blueberry extract in the form of Vaccinium myrtillus fruit extract is present at about 0.08%, pomegranate extract in the form of Punica granatum fruit extract is present at about 0.02%, dipotassium glycyrrhizate is present at about 0.20%, hydroxypropyl tetrahydropyrantriol is present at about 10.5%, all amounts by weight, based on the weight of the composition.

In an embodiment, the disclosure provides a gel-cream emulsion comprising: a blend of skin actives comprising blueberry extract; pomegranate extract; dipotassium glycyrrhizate; and hydroxypropyl tetrahydropyrantriol, wherein blueberry extract in the form of Vaccinium myrtillus fruit extract is present in a range in a range from about 0.01% to about 5%, pomegranate extract in the form of Punica granatum fruit extract is present in a range in a range from about 0.01% to about 5%, dipotassium glycyrrhizate is present in a range in a range from about 0.05% to about 5%, and hydroxypropyl tetrahydropyrantriol is present in a range in a range from about 0.5% to about 15%; and a cosmetic carrier system comprising: water present in a range from about 30% to about 40%; at least one silicone-based emulsifier comprising dimethicone/PEG-10/15 crosspolymer and lauryl PEG-9 polydimethylsiloxyethyl dimethicone, the dimethicone/PEG-10/15 crosspolymer present in a range in a range from about 3% to about 4%, the lauryl PEG-9 polydimethylsiloxyethyl dimethicone present in a range in a range from about 1% to about 1.5%; at least one silicone oil comprising dimethicone present in a range in a range from about 5% to about 25%; at least one branched, liquid alkane comprising isohexadecane present in a range in a range from about 1% to about 15%; at least one hydrating agent present in a range from about 5% to about 30% and comprising glycerin, propylene glycol, ethylhexyl hydroxystearate, and synthetic wax; and at least one additive comprising aluminum starch octenylsuccinate, bismuth oxychloride, cellulose, silica silylate, phenoxyethanol, sorbitol, sodium citrate, magnesium sulfate, and trisodium ethylenediamine disuccinate, all amounts by weight, based on the weight of the composition, wherein the cosmetic composition confers a statistically significant reduction in the expression of at least one gene selected from the group consisting of mitogen-activated protein kinase 5 [MAP3K5], Intraflagellar transport protein 57 [IFT57], Multifunctional ROCO Family Signaling Regulator 1 [MFHAS1], and combinations thereof, and wherein the cosmetic composition is intended to leave on, rinse off, or use as a cleanser.

In some embodiments, blueberry extract in the form of Vaccinium myrtillus fruit extract is present at about 0.08%, pomegranate extract in the form of Punica granatum fruit extract is present at about 0.02%, dipotassium glycyrrhizate is present at about 0.20%, and hydroxypropyl tetrahydropyrantriol is present at about 10.5%, all amounts by weight, based on the weight of the composition. In some embodiments, propylene glycol is present at about 12.54%, by weight, glycerin is present at about 4.4%, synthetic wax is present at about 4.00%, ethylhexyl hydroxystearate is present at about 0.3%; and the at least one additive comprises aluminum starch octenylsuccinate present at about 2.50%, bismuth oxychloride present at about 0.7%, cellulose present at about 0.10%, silica silylate present at about 0.20%, phenoxyethanol present at about 0.70%, sorbitol present at about 0.08%, sodium citrate present at about 0.03%, magnesium sulfate present at about 0.70%, and trisodium ethylenediamine disuccinate present at about 0.11%, all amounts by weight, based on the weight of the composition. In some embodiments the cosmetic composition is an emulsion, a gel, a single phase, or a bi-phase, and wherein the cosmetic composition is in the form of a thick cream, gel-cream, gel, lotion, toner, or serum, and may be provided and intended to leave on, rinse off, or use as a cleanser.

The combination of blueberry extract; pomegranate extract; dipotassium glycyrrhizate; and hydroxypropyl tetrahydropyrantriol demonstrates the effect of statistically significant reduction of protein expression of evaluated genes as demonstrated in ex vivo experiments. Inasmuch as the expression of each of the evaluated proteins is known to adversely affect key cellular process, it is expected that such expression also impacts the quality of skin and contributes to the signs of aging. Accordingly, the cosmetic composition is useful for addressing skin aging, including, but not limited to age related features such as lines, wrinkles, crepiness, and sagging, wherein the benefits that may be conferred by a composition according to the disclosure may include, but are not limited to, one or more of resisting loss of or increasing firmness, resisting formation of or reducing fine lines and wrinkles, resisting loss of or increasing elasticity, and resisting loss of or improving hydration.

Except for the novel combination of actives, other ingredients as mentioned herein may be included or excluded from the composition.

These and other aspects of the invention are set out in the appended claims and described in greater detail in the detailed description of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### DETAILED DESCRIPTION OF THE INVENTION

The disclosure relates to a cosmetic composition to combat skin aging that includes a novel blend of skin actives comprising blueberry extract, pomegranate extract, dipotassium glycyrrhizate, and hydroxypropyl tetrahydropyrantriol in a cosmetic carrier system comprising water, wherein the cosmetic composition demonstrates the unexpected effect of a significant reduction of expression of proteins from genes known to be involved in the skin aging process. The skin actives are provided in cosmetic carrier system that may be an emulsion, for example a silicone emulsion having a thick cream texture as exemplified herein, or other type of emulsion, a gel, gel-cream, a single phase, or a bi-phase, and wherein the cosmetic composition is a leave on treatment in the form of a gel-cream, cream, gel, lotion, toner, or serum, and may be provided and intended to leave on, rinse off, or use as a cleanser.

In some embodiments, the cosmetic composition according to the disclosure is a water-in-oil (as exemplified herein, a silicone oil) emulsion. A water-in-oil emulsion is sometimes referred to as a reverse emulsion in comparison with the more common type of oil-in-water emulsion skin cleanser formulations. The cosmetic composition has a gel-cream texture that demonstrates medium to long playtime, providing a pleasant feel when applied.

In formulating the inventive composition, the inventors sought to improve on the benefits of existing compositions that are known to confer benefits in reducing the extent of biological molecule glycation which is associated with adversely contributing to skin aging. Glycation is a biochemical process that occurs when a sugar molecule, such as glucose or fructose, spontaneously bonds to a protein or lipid molecule without the controlling action of an enzyme. This reaction results in the formation of a class of compounds known as Advanced Glycation End-products (AGEs), for example, pyrraline, carboxymethyllysine, pentosidine, crossline, N^{ε}(2-carboxyethyl)lysine (CEL), glyoxallysine dimer (GOLD), methylglyoxallysine dimer (MOLD), 3DG-ARG imidazolone, versperlysines A, B and C, threosidine or the advanced glycosylation end products (or AGEs). AGEs are known to adversely affect the signs of skin aging, among other processes. The phenomenon of glycation progresses with age.

In particular, the inventors sought to provide a cosmetics skin care composition that includes ingredients associated with combatting AGEs and conferring additional benefits that include reducing the effects of tissue processes that can promote stress response, inhibit cell proliferation, promote cell apoptosis and promote inflammatory response. As described herein, the inventive composition provided benefits that include impacting by downregulation to reduce the expression of proteins that cause or contribute to the above listed tissue processes, and thus confer anti-aging and soothing benefits to skin. These benefits are obtained in the inventive composition which includes hydroxypropyl tetrahydropyrantriol (also referred to herein as Proxylane), Blueberry extract (also referred to herein as Vaccinium Myrtillus Fruit Extract), and Pomegranate extract (also referred to herein as Punica granatum Fruit Extract). Among the ingredients tested, the inventors combined blueberry extract, pomegranate extract, dipotassium glycyrrhizate, and hydroxypropyl tetrahydropyrantriol, variously and together, and with either rhamnose, gentiana, or dipotassium glycyrrhizate, where at least dipotassium glycyrrhizate specifically has been reported in the literature as having anti-glycation benefits. But quite unexpectedly, the results of proteomic studies revealed that addition of dipotassium glycyrrhizate to blueberry extract, pomegranate extract, and hydroxypropyl tetrahydropyrantriol to provide the claimed novel combination of blueberry extract, pomegranate extract, dipotassium glycyrrhizate, and hydroxypropyl tetrahydropyrantriol resulted in a significant reduction of protein expression products of genes known to be involved in the skin aging process, as further described in the Examples herein. The proteomics studies show that blueberry extract, pomegranate extract, and hydroxypropyl tetrahydropyrantriol, alone or in various sub combinations with each did not have a significant positive effect on reducing gene expression, whereas in contrast, the claimed combination of blueberry extract, pomegranate extract, dipotassium glycyrrhizate, and hydroxypropyl tetrahydropyrantriol, as shown herein, demonstrated a statistically significant impact on peptide expression as shown in proteomic studies of gene products known to be involved in the skin aging process. Further, rhamnose or gentiana, alone or in combinations with blueberry extract, pomegranate extract, and hydroxypropyl tetrahydropyrantriol, likewise did not have a significant positive effect on reducing peptide expression as shown in proteomic studies of gene products known to be involved in the skin aging process (data not included here).

The cosmetic composition demonstrates the effect of statistically significant reduction of protein expression of evaluated genes as demonstrated in ex vivo experiments. Inasmuch as the expression of each of the evaluated proteins is known to adversely affect key cellular process, it is expected that such expression also impacts the quality of skin and contributes to the signs of aging. Accordingly, the cosmetic composition is useful for addressing skin aging, including, but not limited to age related features such as lines, wrinkles, crepiness, and sagging, wherein the benefits that may be conferred by a composition according to the disclosure may include, but are not limited to, one or more of resisting loss of or increasing firmness, resisting formation of or reducing fine lines and wrinkles, resisting loss of or increasing elasticity, and resisting loss of or improving hydration.

### Compositions

In various embodiments, the cosmetic composition comprises blueberry extract, pomegranate extract, dipotassium glycyrrhizate, and hydroxypropyl tetrahydropyrantriol, and other optional ingredients comprising a suitable cosmetic carrier. The cosmetic carrier may comprise any one or more ingredients selected from, but not limited to, water, oils, hydrating agents, emulsifiers, surfactants, water based solvents, oil based solvents, additional skin actives and other additives as described herein. In some embodiments, certain ingredients as described herein as optional may be expressly excluded or present in de minimis amounts.

### Blueberry Extract

In various embodiments, the cosmetic composition includes blueberry extract (Vaccinium Myrtillus Fruit Extract).

The cosmetic composition comprises the blueberry extract present in a range from about 0.01% to about 5%, or from about 0.015% to about 1%, or from about 0.01% to about 0.5%, or from about 0.02% to about 0.5%, or about 0.02%, or any suitable combination, sub-combination, range, or sub-range thereof by weight, based on the weight of the cosmetic composition. One of ordinary skill in the art, however, will appreciate that other ranges are within the scope of the invention.

In some embodiments, Vaccinium Myrtillus Fruit Extract is present in a range from about 0.01% to about 5% by weight, based on the weight of the cosmetic composition, and in some embodiments, present at about 0.02% by weight, based on the weight of the cosmetic composition.

Thus, blueberry extract is present by weight, based on the total weight of the cosmetic composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, to about 5 percent, including increments and ranges therein and there between.

### Pomegranate Extract

In various embodiments, the cosmetic composition includes pomegranate extract (Punica granatum Fruit Extract).

The cosmetic composition comprises pomegranate extract present in a range from about 0.01% to about 5%, or from about 0.015% to about 1%, or from about 0.02% to about 0.5%, or from about 0.01% to about 0.1%, or about 0.02%, or any suitable combination, sub-combination, range, or sub-range thereof by weight, based on the weight of the cosmetic composition. One of ordinary skill in the art, however, will appreciate that other ranges are within the scope of the invention.

In various embodiments, Punica granatum Fruit Extract, present in a range from about 0.01% to about 5% by weight, based on the weight of the cosmetic composition, and in some embodiments, present at about 0.08% by weight, based on the weight of the cosmetic composition.

Thus, pomegranate extract is present by weight, based on the total weight of the cosmetic composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, to about 5 percent, including increments and ranges therein and there between.

### Dipotassium Glycyrrhizate

In various embodiments, the cosmetic composition includes dipotassium glycyrrhizate.

The cosmetic composition comprises dipotassium glycyrrhizate from about 0.05% to about 5%, or from about 0.08% to about 3%, or from about 0.08% to about 1.5%, or from about 0.08% to about 0.3%, or about 0.2%, or any suitable combination, sub-combination, range, or sub-range thereof by weight, based on the weight of the cosmetic composition. One of ordinary skill in the art, however, will appreciate that other ranges are within the scope of the invention.

In various embodiments, Dipotassium Glycyrrhizate is present in a range from about 0.05% to about 5% by weight, based on the weight of the cosmetic composition, and in some embodiments, present at about 0.20% by weight, based on the weight of the cosmetic composition.

Thus, dipotassium glycyrrhizate is present by weight, based on the total weight of the cosmetic composition, from about 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, to about 5 percent, including increments and ranges therein and there between.

### Hydroxypropyl Tetrahydropyrantriol

In various embodiments, the cosmetic composition includes Hydroxypropyl Tetrahydropyrantriol, also known as Proxylane. Proxylane is a green chemistry ingredient derived from beech wood that stimulates proteoglycans production in human skin.

The cosmetic composition comprises hydroxypropyl tetrahydropyrantriol from about 0.05% to about 15%, or from about 0.5% to about 15%, or from about 8% to about 12%, or about 10.5%, or any suitable combination, sub-combination, range, or sub-range thereof by weight, based on the weight of the cosmetic composition. One of ordinary skill in the art, however, will appreciate that other ranges are within the scope of the invention.

In various embodiments, hydroxypropyl tetrahydropyrantriol is present in a range from about 0.5% to about 15% by weight, based on the weight of the cosmetic composition, and in some embodiments, present at about 10.5% by weight, based on the weight of the cosmetic composition.

Thus, hydroxypropyl tetrahydropyrantriol is present by weight, based on the total weight of the cosmetic composition, from about 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9, 10, 11, 12, 13, 14, to about 15 percent, including increments and ranges therein and there between.

### Exemplary Formulation: Emulsion Embodiment

### Silicone-Based Emulsifiers

In some embodiments the cosmetic composition may comprise at least one silicone-based emulsifier. In some embodiments, the cosmetic composition comprises more than one silicone-based emulsifier. In some embodiments, the cosmetic composition includes two or more silicone-based emulsifiers, wherein the two silicone-based emulsifiers may differ from one another in that one emulsifier varies in a property such as having a greater chemical molecular weight than the other emulsifier.

In some embodiments, suitable silicone-based emulsifiers may be chosen from silicone emulsifiers that include functional groups chosen from polyglyceryl, polyethylene glycol, or polypropylene glycol, wherein such silicone-based emulsifiers may be chosen from dimethicone/PEG-10/15 crosspolymer, dimethicone/polyglycerin-3 crosspolymer, polyether-modified crosslinked silicone polymers in dimethicone, including, for example, but not limited to PEG-15/lauryl polydimethylsiloxyethyl dimethicone crosspolymer.

In some embodiments, suitable silicone-based emulsifiers may be chosen from alkyldimethicone copolyol type and dimethicone copolyol type silicone emulsifiers. The at least one silicone-based emulsifier may be chosen from dimethicone/PEG-10/15 crosspolymer, lauryl PEG-9 polydimethylsiloxyethyl dimethicone, dimethicone/polyglycerin-3 crosspolymer, cetyl PEG/PPG-10/1 dimethicone, PEG-18/PPG-18 dimethicone, PEG-10 dimethicone, PEG/PPG-18/18 methicone, lauryl PEG/PPG-18/18 methicone, PEG-9 polydimethylsiloxyethyl dimethicone, and combinations thereof.

In some particular embodiments according to the disclosure, the cosmetic composition comprises a silicone-based emulsifier comprising dimethicone/PEG-10/15 crosspolymer, alone or in combination with lauryl PEG-9 polydimethylsiloxyethyl dimethicone.

In some embodiments a particular combination of Dimethicone, Lauryl PEG-9 Polydimethylsiloxyethyl Dimethicone, and Dimethicone/PEG-10/15 Crosspolymer at ~20.7 wt % provides a robust cream having an inverse emulsion that maintains stability at industrial scale conditions, including at temperatures at least up to 40° C., wherein stability is demonstrated by absence of fluid pooling during storage at temperatures at least up to 40° C.

In some embodiments, the cosmetic composition may include the following ingredients in amounts based on the total weight of the cosmetic composition that include: Dimethicone present at about 15.64% by weight, based on the total weight of the cosmetic composition, Lauryl PEG-9 Polydimethylsiloxyethyl Dimethicone present at about 1.12% by weight, based on the total weight of the cosmetic composition, and PEG-10/15 Crosspolymer present at about 3.75% by weight, based on the total weight of the cosmetic composition.

In various embodiments, the at least one silicone-based emulsifier is present with at least one silicone oil, such as but not limited to dimethicone, which may be present in a range from about 5% to about 25%, or about 5% to about 20%, or about 12% to about 18%, or about 15%.

In various embodiments, each of the at least one silicone-based emulsifier is present in the cosmetic composition at a concentration, from about 0.1% to about 20%, or from about 0.2% to about 15%, or from about 0.5% to about 15%, or from about 0.3% to about 10%, or from about 1% to about 5%, or from about 2% to about 4%, or any suitable combination, sub-combination, range, or sub-range thereof by weight, based on the weight of the cosmetic composition. In some embodiments, the cosmetic composition comprises more than one silicone-based emulsifier, the combination thereof present in the cosmetic composition at a concentration, from about 0.2% to about 20%, and each of the more than one present in the cosmetic composition at a concentration from about 0.1% to about 20%, or any suitable combination, sub-combination, range, or sub-range thereof by weight, based on the weight of the cosmetic composition. One of ordinary skill in the art, however, will appreciate that other ranges are within the scope of the invention.

In some representative examples, a silicone-based emulsifier comprising dimethicone/PEG-10/15 crosspolymer may be present in the cosmetic composition in a range from about 3% to about 4% or at about 3.75% together with lauryl PEG-9 polydimethylsiloxyethyl dimethicone present in the cosmetic composition in a range from about 1% to about 1.5% or at about 1.12%.

Thus, in various embodiments, each of the at least one silicone-based emulsifier is present in a composition according to the disclosure from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19 to about 20 percent by weight, including increments and ranges there between. And in various embodiments, each of the at least one silicone oil is present in a composition according to the disclosure from about 05, 6, 7, 8, 9, 10, 11, 12, 13, 14, 15, 16, 17, 18, 19, 20, 21, 22, 23, 24 to about 25 percent by weight, including increments and ranges there between.

### Branched, Liquid Alkane

In some embodiments the cosmetic composition may comprise at least one branched, liquid alkane. In some embodiments, the cosmetic composition comprises more than one branched, liquid alkane. In some embodiments, the branched, liquid alkane has a carbon chain length of C12 to C20.

In some embodiments, the branched, liquid alkane may be chosen from those with a carbon chain length of from C12 to C20, from C13-C15, from C15 to C19, and C12, C13, C14, C15, C16, C17, C18, C19 and C20. In some particular embodiments, the branched, liquid alkane may be chosen from isoparaffins, for instance isohexadecane, isododecane (also known as 2,2,4,4,6-pentamethylheptane), isodecane, and C15-19 alkane.

In some particular embodiments the at least one branched, liquid alkane comprises isohexadecane.

In some embodiments, the cosmetic composition may include the following ingredients in amounts based on the total weight of the cosmetic composition that include: Isohexadecane present at about 5.00% by weight, based on the total weight of the cosmetic composition.

In various embodiments, the at least one branched, liquid alkane is present in the cosmetic composition at a concentration, from about 1% to about 15%, or from about 2% to about 10%, or from about 2% to about 5% or any suitable combination, sub-combination, range, or sub-range thereof by weight, based on the weight of the cosmetic composition. In some embodiments, the cosmetic composition comprises more than one branched, liquid alkane, the combination thereof present in the cosmetic composition at a concentration, from about 5% to about 10%, and each of the more than one present in the cosmetic composition at a concentration from about 2% to about 5% or any suitable combination, sub-combination, range, or sub-range thereof by weight, based on the weight of the cosmetic composition. One of ordinary skill in the art, however, will appreciate that other ranges are within the scope of the invention.

Thus, in various embodiments, each of the at least one branched, liquid alkane is present in a composition according to the disclosure from about 1, 2, 3, 4, 5, 6, 7, 8, 9, 10, 11, 12, 13, 14 to about 15 percent, by weight, including increments and ranges therein and there between.

### Hydrating Agent

In some embodiments the cosmetic composition may comprise at least one hydrating agent.

In some embodiments, the cosmetic composition comprises more than one hydrating agent.

In some embodiments, suitable hydrating agents that may be used according to the disclosure may be chosen one or more of polyols, including, for example, glycerin, propylene glycol, dipropylene glycol, glycerol, glycols, such as caprylyl glycol, butylene glycol, isoprene glycol, hexylene glycol and polyethylene glycols, monoethylene glycol, diethylene glycol, diethylene glycol, diethylene glycol, hexylene glycol; ethylhexyl hydroxystearate; synthetic wax; glycol ethers such as monopropylene, dipropylene and tripropylene glycol alkyl(C1-C4)ethers; squalane; triacetin; sugars, such as glucose, xylitol, maltitol, sorbitol, sucrose pentaerythritol, inositol; pyrrolidone carboxylic acid (PCA); lactic acid; lithium chloride; acetamide MEA; sodium lactate; urea; dicyanamide; hyaluronic acid; aloe vera; honey; seaweed extract; and combinations thereof.

In some embodiments, the hydrating agent comprises one or a combination of glycerin, propylene glycol, dipropylene glycol, ethylhexyl hydroxystearate, and synthetic wax.

In some embodiments, the hydrating agent comprises glycerin, propylene glycol, ethylhexyl hydroxystearate, and synthetic wax.

In some embodiments, the cosmetic composition may include the following ingredients in amounts based on the total weight of the cosmetic composition that include: Synthetic Wax present at about 4.00% by weight, based on the total weight of the cosmetic composition, and Ethylhexyl Hydroxystearate present at about 0.3% by weight, based on the total weight of the cosmetic composition.

In some embodiments, the cosmetic composition may include the following ingredients in amounts based on the total weight of the cosmetic composition that include: Propylene Glycol present at about 12.54% by weight, based on the total weight of the cosmetic composition, Glycerin present at about 4.4% by weight, based on the total weight of the cosmetic composition, and Dipropylene Glycol present at about 0.08% by weight, based on the total weight of the cosmetic composition.

In various embodiments, the at least one hydrating agent is present in a range from about 5% to about 30% by weight of the cosmetic composition, or from about 5% to about 25%, or from about 10% to about 20%, and in some embodiments, about 15%, or any suitable combination, sub-combination, range, or sub-range thereof by weight, based on the weight of the cosmetic composition. In some embodiments, the cosmetic composition comprises more than one hydrating agent, the combination thereof present in the cosmetic composition at a concentration, from about 5% to about 30%, and each of the more than one present in the cosmetic composition at a concentration from about 5% to about 25%, and in some embodiments, about 15% or any suitable combination, sub-combination, range, or sub-range thereof, by weight, based on the weight of the cosmetic composition. One of ordinary skill in the art, however, will appreciate that other ranges are within the scope of the invention.

Thus, in various embodiments, each of the at least one emollient ester, ether, or carbonate is present in a composition according to the disclosure from about 5, 6, 7, 8, 9, 10, 11, 12, 13, 14, 15, 16, 17, 18, 19, 20, 21, 22, 23, 24, 25, 26, 27, 28, 29, to about 30 percent, by weight, including increments and ranges therein and there between.

### Water

In some embodiments, the cosmetic composition may include water. In accordance with such embodiments, water is present in the cosmetic compositions in a range from about 20% to about 95%, or from about 25% to about 85%, or from about 30% to about 75%, or from about 30% to about 40%, and in some embodiments, about 35% or any suitable combination, sub-combination, range, or sub-range thereof by weight, based on the weight of the cosmetic composition.

In some embodiments, the cosmetic composition may include water present in a range from about 20% to about 95% based on the total weight of the cosmetic composition. In some embodiments, the cosmetic composition may include water present in a range from about 25% to about 40% based on the total weight of the cosmetic composition, and in some embodiments about 35% based on the total weight of the cosmetic composition.

One of ordinary skill in the art, however, will appreciate that other ranges are within the scope of the invention. Thus, water is present, by weight, based on the total weight of the cosmetic composition, from about 20, 21, 22, 23, 24 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, to about 95 percent, by weight, including increments and ranges therein and there between. One of ordinary skill in the art, however, will appreciate that other ranges are within the scope of the invention.

The water used may be sterile demineralized water and/or a floral water such as rose water, cornflower water, chamomile water or lime water, and/or a natural thermal or mineral water such as, for example: water from Vittel, water from the Vichy basin, water from Uriage, water from La Roche Posay, water from La Bourboule, water from Enghien-les-Bains, water from Saint Gervais-les-Bains, water from Neris-les-Bains, water from Allevar-les-Bains, water from Digne, water from Maizieres, water from Neyrac-les-Bains, water from Lons-le-Saunier, water from Eaux Bonnes, water from Rochefort, water from Saint Christau, water from Les Fumades, water from Tercis-les-Bains or water from Avene. The water phase may also comprise reconstituted thermal water, which is to say a water comprising trace elements such as zinc, copper, magnesium, etc., reconstituting the characteristics of a thermal water.

The pH of the cosmetic composition may be adjusted prior to combining the oil phase with the water phase to avoid the practical difficulty with measuring pH in an internal water phase of the water-in-oil emulsion. Accordingly, the pH of the water phase prior to emulsification can be adjusted with pH adjusters to a pH in a range from about 2 to about 12, and in some embodiments, is from about 3 to about 11, and from about 4 to about 9, and from about 5 to about 8, and in some embodiments about 5.5. The pH of the water phase can be adjusted by addition of a base (organic or inorganic) to the cosmetic composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

### Additional Ingredients/Additives

In some embodiments, the cosmetic composition may include at least one additional ingredient selected from, but not limited to, water, oils, hydrating agents, thickeners, emulsifiers, surfactants, water based solvents, oil based solvents, additional skin actives, fillers, preservatives, chelating agents, pH adjusters, dispersants, pigments, effect pigments, dyes, colorants, powders, opacifiers, fragrances, and combinations thereof.

In some embodiments wherein the cosmetic composition comprises an emulsion, and may include the following ingredients in amounts based on the total weight of the cosmetic composition that include: adenosine present at about 0.04% by weight, based on the total weight of the cosmetic composition, tocopherol present at about 0.003% by weight, based on the total weight of the cosmetic composition, and methyldihydrojasmonate present at about 0.5% by weight, based on the total weight of the cosmetic composition (the foregoing combined at ~0.543 wt %), aluminum starch octenylsuccinate present at about 2.50% by weight, based on the total weight of the cosmetic composition, bismuth oxychloride present at about 0.7% by weight, based on the total weight of the cosmetic composition, cellulose present at about 0.10% by weight, based on the total weight of the cosmetic composition, silica silylate present at about 0.20% by weight, based on the total weight of the cosmetic composition, phenoxyethanol present at about 0.70% by weight, based on the total weight of the cosmetic composition, sorbitol present at about 0.08% by weight, based on the total weight of the cosmetic composition, sodium citrate present at about 0.03% by weight, based on the total weight of the cosmetic composition, magnesium sulfate present at about 0.70% by weight, based on the total weight of the cosmetic composition, and trisodium ethylenediamine disuccinate present at about 0.11% by weight, based on the total weight of the cosmetic composition.

In some embodiments, the cosmetic composition may include the following ingredients in amounts based on the total weight of the cosmetic composition that include: aluminum starch octenylsuccinate present at about 2.50% by weight, based on the total weight of the cosmetic composition, bismuth oxychloride present at about 0.7% by weight, based on the total weight of the cosmetic composition, cellulose present at about 0.10% by weight, based on the total weight of the cosmetic composition, and silica silylate present at about 0.20% by weight, based on the total weight of the cosmetic composition.

In some embodiments, the cosmetic composition may include the following ingredients in amounts based on the total weight of the cosmetic composition that include: phenoxyethanol present at about 0.70% by weight, based on the total weight of the cosmetic composition, sorbitol present at about 0.08% by weight, based on the total weight of the cosmetic composition, sodium citrate present at about 0.03% by weight, based on the total weight of the cosmetic composition, magnesium sulfate present at about 0.70% by weight, based on the total weight of the cosmetic composition, and trisodium ethylenediamine disuccinate present at about 0.11% by weight, based on the total weight of the cosmetic composition.

In some embodiments, the cosmetic composition may include the following ingredients in amounts based on the total weight of the cosmetic composition that include: Fragrance present at about 0.10% by weight, based on the total weight of the cosmetic composition.

### Water-Soluble Solvents

In some embodiments, the cosmetic composition may include at least one water-soluble solvent. The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-C30, C1-C15, C1-C10, or C1-C4 alcohols), organic solvents, polyols, glycols, or mixtures thereof.

In some particular embodiments according to the disclosure, when present, a water-soluble solvent may include glycerin, propylene glycol, dipropylene glycol, or combinations thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylene glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or mixtures thereof.

In various embodiments the amount of the at least one water-soluble solvent, when present, is from about 0.1% to about 25%, or from about 0.1% to about 2%, or from about 0.1% to about 1%, or from about 0.1% to about 0.8%, or from about 0.1% to about 0.5%, or from about 1% to about 20%, or from about 1% to about 10%, or from about 2% to about 8%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one water soluble solvent, each water soluble solvent present in an amount as set forth herein above, wherein each different water soluble solvent may be present within one of the ranges selected from the ranges set forth herein above.

Thus, each one or combination of water-soluble solvents, when present, may be present by weight, based on the total weight of the cosmetic composition, from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24 to about 25 weight percent, including increments and ranges therein and there between.

### Thickener

In some embodiments, the cosmetic composition may include one or more thickener. The thickener may be synthetic, mineral, or organic in nature, and may comprise a particulate of any shape. More generally, by way of non-limiting example, thickeners may be chosen from xanthan gum, cellulose gum, acacia senegal gum, guar gum, sclerotium gum, dehydroxanthan gum, gellan gum, agar, algin, talc, mica, silica, silica surface-treated with a hydrophobic agent, fumed silica, kaolin, clay/smectite mineral (Argilla extract) polyamide (NYLON) powders (e.g. ORGASOL from Atochem), polyurethane powders, poly-β-alanine powder and polyethylene powder, powders of tetrafluoroethylene polymers (TEFLON), lauroyllysine, starch, boron nitride, hydrophobic silica aerogel particles, hollow polymer microspheres such as those of polyvinylidene chloride/acrylonitrile, for instance EXPANCEL (Nobel Industrie) or of acrylic acid copolymers (POLYTRAP from the company Dow Corning) and silicone resin microbeads (TOSPEARLS from Toshiba, for example), elastomeric polyorganosiloxane particles, precipitated calcium carbonate, magnesium carbonate, magnesium hydrogen carbonate, hydroxyapatite, hollow silica microspheres (SILICA BEADS from Maprecos), glass or ceramic microcapsules, and metal soaps derived from organic carboxylic acids containing from 8 to 22 carbon atoms and preferably from 12 to 18 carbon atoms, for example zinc stearate, magnesium stearate, lithium stearate, zinc laurate, magnesium myristate and combinations thereof.

In some embodiments, one or more thickener may be present in the cosmetic composition from about 0.01% to about 10%, or from about 0.01% to about 5%, or from about 0.01% to about 1%, or from about 0.01% to about 0.2%, or from about 0.01% to about 0.09%, or from about 0.02% to about 0.08%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one thickener, each thickener present in an amount as set forth herein above, wherein each different thickener may be present within one of the ranges selected from the ranges set forth herein above.

Thus, each one or combination of thickeners, when present, may be present by weight, based on the total weight of the cosmetic composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, to about 10 percent, including increments and ranges therein and there between.

### Surfactant

In some embodiments, the cosmetic composition may include one or more surfactants. In some particular embodiments, the cosmetic composition may include an emulsion that comprises one or more surfactants, alone or in combination, with one or more oil (as described below).

The one or more surfactant in the cosmetic composition may be present in an amount ranging from about 0.1% to about 5%, or from about 0.1% to about 1%, or from about 0.2% to about 0.8%, or from about 0.3% to about 0.5%. In at least certain exemplary embodiments, the surfactant is present in an amount less than about 1%, or less than about 0.8%. In some embodiments, the cosmetic composition includes more than one surfactant, each surfactant present in an amount as set forth herein above, wherein each different surfactant may be present within one of the ranges selected from the ranges set forth herein above.

Thus, each one or combination of surfactants, when present, may be present by weight, based on the total weight of the cosmetic composition, from about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, to about 5 percent, including increments and ranges therein and there between.

### Oil

In some embodiments, the cosmetic composition may include one or more oils. In some embodiments, the cosmetic composition may include an emulsion that comprises one or more oils, alone or in combination, with one or more surfactant (as described above).

As used herein, oil refers to any nonpolar compound that is a liquid at 25° C. and is hydrophobic and lipophilic. Oil that is suitable for use herein may be volatile or non-volatile. The term “volatile oil” relates to oil that is capable of evaporating on contact with the skin or a keratin fiber in less than one hour, at room temperature and atmospheric pressure. The volatile oil(s) are liquid at room temperature and have a non-zero vapor pressure, at room temperature and atmospheric pressure, ranging in particular from 0.13 Pa to 40 000 Pa (10-3 to 300 mmHg). The term “non-volatile oil” relates to oil which remains on the skin or the keratin fiber, at room temperature and atmospheric pressure, for at least several hours and which in particular has a vapor pressure of less than 10-3 mmHg (0.13 Pa).

In some embodiments, by way of non-limiting example, the one or more oil in the cosmetic composition may be chosen from botanical and essential oils, such as Helianthus Annuus Seed Oil, Mentha Piperita Oil, Rosmarinus Officinalis (rosemary) Leaf Oil Pelargonium Graveolens flower oil, Citrus aurantium dulcis (orange) peel oil, Menthe Viridis (spearmint) leaf oil, Citrus aurantifolia (lime) oil, Melaleuca Alternifolia (tea tree) leaf oil, Citrus Grandis (grapefruit) peel oil, Citrus Medica Limonum (lemon) peel oil, rose flower oil, eucalyptus globulus leaf oil, and combinations thereof.

The term “silicone oil” relates to oil comprising at least one silicon atom, and especially at least one Si—O group. The term “fluoro oil” relates to oil comprising at least one fluorine atom. The term “hydrocarbon-based oil” relates to oil comprising mainly hydrogen and carbon atoms. Hydrocarbon-based oil may be animal hydrocarbon-based oil, plant hydrocarbon-based oil, mineral hydrocarbon-based oil, or a synthetic hydrocarbon-based oil. Further, suitable oil may be a mineral hydrocarbon-based oil, a plant hydrocarbon-based oil, or a synthetic hydrocarbon-based oil.

### Silicone Oils

The cosmetic composition may include one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the cosmetic composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

Other examples of silicone oils that may be mentioned include volatile linear or cyclic silicone oils, especially those with a viscosity 8 centistokes (8×106 m2/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

### Fluoro Oils

The cosmetic composition may include one or more fluoro oils. For example, the one or more fluoro oil may be selected from the group consisting of perfluoromethylcyclopentane, perfluoro-1,3-dimethylcyclohexane, dodecafluoropentane, tetradecafluorohexane, bromoperfluorooctyl, nonafluoromethoxybutane, nonafluoroethoxyisobutane and 4-trifluoromethylperfluoromorpholine. Volatile fluoro oils, such as nonafluoromethoxybutane, decafluoropentane, tetradecafluorohexane, dodecafluoropentane, may also be used.

### Hydrocarbon-Based Oils

The cosmetic composition may include one or more hydrocarbon-based oils. For example, the hydrocarbon-based oil may be a saturated hydrocarbon, an unsaturated hydrocarbon, lipids, triglycerides, a natural oil, and/or a synthetic oil. In some embodiments, the cosmetic composition may include a synthetic oil selected from the group consisting of hydrogenated polyisobutene and hydrogenated polydecene. A hydrocarbon-based oil may be a non-volatile hydrocarbon-based, such as:
(i) hydrocarbon-based oils of plant origin, such as glyceride triesters, which are generally triesters of fatty acids and of glycerol, the fatty acids of which can have varied chain lengths from C4 to C24, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil, and musk rose oil.
(ii) synthetic ethers containing from 10 to 40 carbon atoms;
(iii) linear or branched hydrocarbons of mineral or synthetic origin, such as petroleum jelly, polydecenes, hydrogenated polyisobutene such as Parleam, and 40 squalane;
(iv) synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms on condition that R+R′ is ÿ 10, for instance Purcellin oil (cetearyl octanoate), isopropyl myristate, isopropyl palmitate, C12-C15 alkyl benzoate, such as the product sold under the trade name Finsolv TN® or Witconol TN® by Witco or Tegosoft TN® by Evonik Goldschmidt, 2-ethylphenyl benzoate, such as the commercial product sold under the name X-Tend 226 by ISP, isopropyl lanolate, hexyl laurate, diisopropyl adipate, isononyl isononanoate, oleyl erucate, 2-ethylhexyl palmitate, isostearyl isostearate, diisopropyl sebacate, such as the product sold under the name of “Dub Dis” by Stearinerie Dubois, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C12-C13 alkyl) tartrates, such as those sold under the name Cosmacol ETI® by Enichem Augusta Industriale, and also di(linear C14-C15 alkyl) tartrates, such as those sold under the name Cosmacol ETL® by the same company; or acetates;
(v) fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
(vi) higher fatty acids, such as oleic acid, linoleic acid, or linolenic acid;
(vii) carbonates, such as dicaprylyl carbonate, such as the product sold under the name Cetiol CC® by Cognis;
(viii) fatty amides, such as isopropyl N-lauroyl sarcosinate, such as the product sold under the trade name Eldew SL 205® from Ajinomoto; and
(ix) essential oils selected from the group consisting of sunflower oil, sesame oil, peppermint oil, macadamia nut oil, tea tree oil, evening primrose oil, sage oil, rosemary oil, coriander oil, thyme oil, pimento berries oil, rose oil, anise oil, balsam oil, bergamot oil, rosewood oil, cedar oil, chamomile oil, sage oil, clary sage oil, clove oil, cypress oil, eucalyptus oil, fennel oil, sea fennel oil, frankincense oil, geranium oil, ginger oil, grapefruit oil, jasmine oil, juniper oil, lavender oil, lemon oil, lemongrass oil, lime oil, mandarin oil, marjoram oil, myrrh oil, neroli oil, orange oil, patchouli oil, pepper oil, black pepper oil, petitgrain oil, pine oil, rose otto oil, rosemary oil, sandalwood oil, spearmint oil, spikenard oil, vetiver oil, wintergreen oil, and ylang ylang.

Hydrocarbon-based oils may be glyceride triesters and in particular to caprylic/capric acid triglycerides, synthetic esters and in particular isononyl isononanoate, oleyl erucate, C12-C15 alkyl benzoate, 2-ethylphenyl benzoate and fatty alcohols, such as octyldodecanol. As volatile hydrocarbon-based oils, mention is made of hydrocarbon-based oils containing from 8 to 16 carbon atoms and in particular of branched C8-C16 alkanes, such as C8-C16 isoalkanes of petroleum origin (also known as isoparaffins), such as isododecane (also known as 2,2,4,4,6-pentamethylheptane), isodecane or isohexadecane, the oils sold under the Isopar or Permethyl trade names, branched C C8-C16 esters, and isohexyl neopentanoate.

In some embodiments, the cosmetic composition may include one or more oils such as from those described herein above, and from oils that may be selected from branched or linear, liquid alkane with carbon chain length of C11 to C20. In various embodiments, liquid alkanes may be selected from those with a carbon chain length of from C11 to C20. The liquid alkanes may be selected from those with a carbon chain length of from C11 to C20, or from C15 to C19, or one of C, 11, C12, C13, C14, C15, C16, C17, C18 to C19. In some embodiments, suitable liquid alkanes that may be used according to the disclosure include hydrocarbon-based oils containing from 8 to 16 carbon atoms, and especially branched C8-C16 alkanes such as C8-C16 isoalkanes.

In some embodiments, the cosmetic composition may include one or more oils selected from polar emollients selected from esters, triglycerides, ethers, carbonates, alcohols, oils, butters, fatty acids, and their combinations thereof. In various embodiments, the polar emollients may be selected from those with a molecular weight of 400 g/mol or less. More, generally, the polar emollient may have a molecular weight in the range from about 50 g/mol % to about 350 g/mol.

In some embodiments, the cosmetic composition may include polar emollients that include those derived from C12-C50 fatty acids, preferably C16-C22 saturated fatty acids, and monohydric alcohols. In some embodiments, such esters may be chosen from isopropyl myristate, methyl palmitate, isopropyl laurate, isopropyl palmitate, ethylhexyl palmitate, ethylhexyl laurate, ethylhexyl oleate, ethylhexyl isononanoate, myristyl myristate, 2-ethylhexyl caprate/caprylate (or octyl caprate/caprylate), 2-ethylhexyl palmitate, isostearyl neopentanoate, isononyl isononanoate, hexyl laurate, esters of lactic acid and of fatty alcohols comprising 12 or 13 carbon atoms, dicaprylyl carbonate and their mixtures.

The one or more oil, when present, may be present in the cosmetic composition from about 0.0001% to about 20% or from about 0.001% to about 0.0010%, or from about 0.003% to about 0.004%, or from about 0.01% to about 0.1%, or from about 0.1% to about 10%, or from about 0.5% to about 20%, or from about 1% to about 10%, or from about 5% to about 10%, or from about 2% to about 7%, or from about 0.5% to about 2%, or from about 0.008% to about 0.01%, or from about 0.1% to about 0.2%, or from about 0.5% to about 2%, or from about or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one oil, each oil present in an amount as set forth herein above, wherein each different oil (such as, for example, plant oils and extracts with oils) may be present within one of the ranges selected from the ranges set forth herein above.

Thus, each one or combination of oils, when present, may be present by weight, based on the total weight of the cosmetic composition, from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2, 3, 4, 5, 6, 7, 8, 9 to about 10 percent, including increments and ranges therein and there between.

### Additional Cosmetic Additives

In some embodiments, the cosmetic composition may include one or more additional actives, for example, cosmetic actives, or other additives such as, for example, oils, pH adjustors, chelating agents, and the like.

In various embodiments, the cosmetic composition may include one or more additional cosmetic actives in addition to the actives as described herein above. In some embodiments, the one or more additional cosmetic active does not adversely affect the properties of the cosmetic composition according to the invention.

Some cosmetic activities may be antimicrobial or preservative in nature, or may include. In some embodiments, one or more optional additional cosmetic active is water soluble.

In some embodiments, actives may be selected from anti-aging ingredients including hydroxypropyl tetrahydropyrantriol, one or more humectant, such as methyl gluceth-20, and one or more vitamin or vitamin derivative, or one of sodium citrate, citric acid, dipotassium glycyrrhizate, Anogeissus Leiocarpus bark extract, hydrolyzed Rhodophycea extract, hydrolyzed linseed extract, Pseudoalteromonas ferment extract, Manilkara Multinervis leaf extract, Lavandula Hybrida oil, Grifola Frondosa fruiting body extract, Plantago Lanceolata leaf extract, Cyathea Medullaris leaf extract, hydrolyzed hyaluronic acid, Malus Domestica fruit cell culture extract, resveratrol, Salicyloyl Phytosphingosine, glaucine, retinol, retinoic acid, and combinations thereof.

In some embodiments, actives may be selected from oily skin treatment ingredients including lens esculenta (lentil) seed extract, sarcosine, Peumus Boldus leaf extract, Crithmum Maritimum extract, niacinamide, salicylic acid, hydroxyethylpiperazine ethane sulfonic acid, Spiraea Ulmaria extract, pentaerythrityl tetra-di-t-butyl hydroxyhydrocinnamate and combinations thereof.

In some embodiments, actives may be selected from skin tone improving ingredients including Ophiopogon Japonicus root extract, Chenopodium quinoa seed extract, Tamarindus indica seed gum, xylitylglucoside (and) anhydroxylitol (and) xylitol, ceramide derivatives, Helianthus annuus (sunflower) seed oil unsaponifiables, myristyl malate phosphonic acid, mannose, 2-oleamido-1,3-octadecanedi and combinations thereof.

In some embodiments, actives may be selected from anti-redness ingredients including madecassoside, saccharide isomerate, palmitoyl tripeptide-8, panthenol, Olea europaea (olive) leaf extract, Mentha Piperita (peppermint) extract, Leontopodium Alpinum extract, dipotassium glycyrrhizate, acetyl dipeptide-1 cetyl ester, acetyl tetrapeptide-15, Boswellia Serrata extract, sodium palmitoyl proline (and) nymphaea alba flower extract and combinations thereof.

In some embodiments, actives may be selected from hydroxy acids.

In some embodiments, actives may be selected from extracts of algae and of planktons, enzymes and coenzymes, ceramides, anti-oxidant compounds, including, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, curcumin, pinoresinol, hydroxytyrosol, cinnamic acid, caffeic acid, p-coumaric acid, baicalin, pine bark, and other extracts and combinations thereof.

In some embodiments, actives may be selected from anti-elastase and anti-collagenase agents, peptides, fatty acid derivatives, steroids; free-radical scavengers, vitamins, and vitamin derivatives, phenylethyl resorcinol, alpha and beta hydroxy acids, and hydrogen peroxide.

In some embodiments, actives may be selected from sunscreen agents (e.g., inorganic sunscreen agent, such as titanium dioxide and zinc oxide and organic sunscreen agents, such as octocrylene, ethylhexyl salicylate, homosalate, and avobenzone); and combinations thereof.

The cosmetic composition can also comprise one or more optional additive used in the cosmetics field which does not adversely affect the properties of the cosmetic composition according to the invention.

Optional additives may be selected from fragrances, pearlescent agents, fillers, powders, preservatives, proteins, protein hydrolysates, vitamins, silicones, odor absorbers and coloring materials; skin conditioners, hydrating agents, anti-microbial components, including, but not limited to, phenoxyethanol, chlorphenesin, capryloyl glycol and sodium salicylate; thickeners and fillers, fruit extracts, for example Pyrus malus (Apple) Fruit Extract, and Aloe Barbadensis Leaf Juice Powder; citric acid, sodium chloride; neutralizing or pH-adjusting agents (e.g., triethylamine (TEA) and sodium hydroxide), and combinations thereof.

In some embodiments, optional additives may be selected from fillers. The fillers may be of mineral or organic origin, natural or synthetic in nature in order to provide oil absorption or optical effects. Oil absorption fillers may impart a matte effect and non-greasy feeling onto the skin. Optical effects fillers may impart a soft-focus/haze/blur effect to the skin, provide the skin with a more uniform appearance, reduce the appearance of skin imperfections or discoloration, or reduce the visibility of pores.

When present, a filler may include vinyl dimethicone/methicone silsesquioxane crosspolymer, ammonium polyacryloyldimethyl taurate, methyl methacrylate crosspolymer, silica silylate, mica, Zea may (corn) starch, magnesium oxide, nylon-12, nylon-66, cellulose, polyethylene, talc, talc (and) methicone, talc (and) dimethicone, perlite, sodium silicate, pumice, PTFE, Ammonium Polyacryloyldimethyl Taurate, polymethyl methacrylate, Oryza sativa (rice) starch, aluminum starch octenylsuccinate, potato starch modified, alumina, calcium sodium borosilicate, magnesium carbonate, hydrated silica, dimethicone/vinyl dimethicone crosspolymer, sodium carboxymethyl starch, bismuth oxychloride, silica silylate, boron nitride, iron oxide, calcium carbonate, calcium sulfate (and) iron oxides, sodium potassium aluminum silicate, silica, silica (and) methicone, silica (and) dimethicone, polysilicone-22, polysilicone-8, polysilicone-11, methyl methacrylate crosspolymer, polymethylsilsesquioxane, methylsilanol/silicate crosspolymer, vinyl dimethicone/methicone silsesquioxane crosspolymer, diphenyl dimethicone/vinyl diphenyl dimethicone silsesquioxane crosspolymer, and styrene/acrylates copolymer.

Although the optional additives are given as examples, it will be appreciated that other optional components compatible with cosmetic applications known in the art may be used.

In various embodiments, one or more additional cosmetic actives, alone or in combination, when present, can be present in the cosmetic composition from about 0.00001% to about 25%, or from about 0.00005% to about 15%, or from about 0.0001% to about 2%, or from about 0.05% to about 1%, or from about 0.02% to about 2%, or from about 0.2% to about 1%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition comprises two, three, four or more actives having some, or all of which may be water soluble. In some embodiments, the cosmetic composition includes more than one additional cosmetic active, each additional cosmetic active present in an amount as set forth herein above, wherein each different additional cosmetic active may be present within one of the ranges selected from the ranges set forth herein above.

In some embodiments, when present, the amount of optional additives present in the cosmetic composition can range from about 0.001% to about 50%, or from about 0.01% to about 30%, or from about 1.5% to about 20%, and from about 5% to about 15%, or from about 0.005% to about 0.1%, or from about 0.05% to about 1%, or from about 0.06% to about 0.075%, or from about 0.1% to about 1%, or from about 0.25% to about 5%, and from about 0.5% to about 3.5%, or from about 0.2% to about 0.8%, or any suitable combination, sub-combination, range, or sub-range thereof by weight, based on the weight of the cosmetic composition. One of ordinary skill in the art, however, will appreciate that other ranges are within the scope of the invention. In some embodiments, the cosmetic composition includes more than one optional component, each optional component present in an amount as set forth herein above, wherein each different optional components may be present within one of the ranges selected from the ranges set forth herein above.

Thus, each one or combination of additives, when present, may be present by weight, based on the total weight of the cosmetic composition, from about 0.00001, 0.00002, 0.00003, 0.00004, 0.00005, 0.00006, 0.00007, 0.00008, 0.00009, 0.0001, 0.0002, 0.0003, 0.0004, 0.0005, 0.0006, 0.0007, 0.0008, 0.0009, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 30, 35, 40, 45 to about 50 percent, including increments and ranges therein and there between.

### EXAMPLES

The following Examples are provided for illustrative purposes only and are not intended to be limiting.

### Example 1: Raw Materials

Raw materials used in the exemplified compositions include, in part, those listed in Table 1.

TABLE 1
Raw Materials (partial list)

| RAW MATERIAL | % ACTIVE |
|---|---|
| Dipotassium Glycyrrhizate | 100 |
| Hydroxypropyl Tetrahydropyrantriol (Proxylane) | 35%, balance propylene glycol, |
| | water |
| Punica Granatum Fruit Extract | 4%, balance glycerin |
| Vaccinium Myrtillus Fruit Extract | 2%, balance water, PG, sorbitol |
| Lauryl PEG-9 Polydimethylsiloxyethyl dimethicone | 99.98%, balance tocopherol |
| Dimethicone/PEG-10/15 Crosspolymer | 25%, balance DPG, dimethicone, |
| | tocopherol, sodium citrate |
| Bismuth Oxychloride | 70%, balance |
| | ethylhexylhydroxystearate |
| Trisodium Ethylenediamine Disuccinate | 37%, balance water |

### Example 2: Proteomics Study

An ex vivo proteomics study was performed using mammalian fibroblast cells (human) to evaluate the effects on gene expression of several genes known to be involved in the skin aging process using single ingredients, combinations of ingredients, and a combination of skin actives according to the disclosure comprising blueberry extract, pomegranate extract, dipotassium glycyrrhizate, and hydroxypropyl tetrahydropyrantriol.

The anti-aging & soothing effect delivered by the composition prepared above was evaluated by means of Proteome study as follows.

### 1. Cell Inoculation

Logarithmic growth phase fibroblast cells were collected, the cells were inoculated into a 24 well plate at 3.2×104 per well, and incubated overnight in a CO2 incubator (37° C., 5% CO2).

### 2. Medication Administration

Each composition prepared above was diluted 96 times with water. When the cell plating rate in the 24 well plate reaches 40%-60%, administration was carried out in groups. A blank control group was added with 2 mL of low sugar DMEM culture medium containing 2% serum per well, and each sample group was added with 2 mL of sample working medium per well. After administration, the 24-well plate was placed in a CO2 incubator (37° C., 5% C02) and incubated for 24 hours. After incubation, 1 ml of PBS was added to each well for 3 washes, each lasting for 3 minutes. After washing, digestion with 0.25% trypsin digestion solution was carried out in the incubator for 3 minutes, then 1 ml of low sugar DMEM culture medium was added to each well. The cell suspension was collected in a 10 ml centrifuge tube, centrifuged, the supernatant was discarded, and protein extraction was carried out.

### 3. Protein Extraction

1) an appropriate amount of 1×Cocktail (protease inhibitor) without SDS protein lysate and with a 0.2 M EDTA (ethylenediaminetetraacetic acid) were added;

2) Crushing and lysing were carried out using an ultrasonic instrument, then centrifuging was carried out at 25000 g*4° C. for 15 minutes, and the supernatant was collected;

3) DTT (dithiothreitol) with a final concentration of 10 mM was added into the supernatant under a water bath at 37° C. for 30 minutes;

4) IAM (iodoacetamide) with a final concentration of 55 mM was added into the supernatant and then placed in a dark room for 45 minutes;

5) 5 times volume of pre-cooled acetone was added into the supernatant, refrigerated overnight at −20° C., centrifuged at 25000 g*4° C. for 15 minutes, and the supernatant was discarded;

6) Air drying and precipitating were carried out, SDS free protein lysate was added, and an automated grinder was used to promote protein dissolution;

7) Centrifuging was carried out at 25000 g at 4° C. for 15 minutes to obtain the supernatant, which is a protein solution.

### 4. Quality Control and Proteomics Testing

### Quality Control:

### (1) SDS-PAGE

10 μg of protein solution from each sample was taken out and mixed with a loading buffer, then heated at 95° C. for 5 minutes, centrifuged at 25,000 g for 5 minutes, the supernatant was taken out and loaded into the wells of a 12% SDS-polyacrylamide gel. Electrophoresis was performed at a constant voltage of 80V for 30 minutes, followed by 120V for 120 minutes. After electrophoresis, the gel was placed in a rapid staining apparatus for 10 minutes, then taken out and the gel image was scanned.

### (2) Bradford Quantitative Assay

In a 96-well microplate, standard protein (0.2 μg/μL BSA) was added in the following volumes to wells A1 to A10: 0, 2, 4, 6, 8, 10, 12, 14, 16, 18 μL. Then pure water was added in the following volumes to each well: 20, 18, 16, 14, 12, 10, 8, 6, 4, 2 μL. Subsequently, 180 μL of Coomassie Brilliant Blue G-250 quantitative working solution was added to each well. The OD595 was measured using a microplate reader, and a linear standard curve was prepared based on OD595 and protein concentration. The protein solution to be tested was diluted by several folds, 180 μL of the quantitative working solution was added to 20 μL of the protein solution, and the OD595 was read. The protein concentration of the sample was calculated based on the standard curve and the sample OD595.

### Proteomics Testing

### (1) Protein Digestion

Trypsin was used to digest the protein sample, and then the resulting peptides were desalted using Strata X columns, followed by vacuum drying.

### (2) High pH RP Separation

Equal amounts of peptides from all samples were mixed and diluted with mobile phase A (5% ACN (Acetonitrile), pH9.8), and liquid phase separation was performed on the samples using a Shimadzu LC-20AD liquid chromatography system under the following conditions: gradient elution with 1 mL/mol, 5% mobile phase B (95% ACN, pH9.8) for 10 min, 5%-35 mobile phase B for 40 min, 35%-95 mobile phase B for 1 min, mobile phase B for 3 min and 5% mobile phase B for 10 min. The elution peaks at a wavelength of 214 nm were monitored and one fraction was collected per minute. Fractions were combined based on the chromatographic elution peak map to obtain ten fractions, and then freeze dried.

### (3) DDA Library Construction and DIA Quantitative Detection (Nano-LC-MS/MS)

After the dried peptide samples were redissolved and centrifuged to take the supernatant, they were separated using a Thermo Scientific Ultimate 3000 UHPLC. The liquid chromatography was connected directly to the mass spectrometer to detect as follows:

### DDA Library Construction and Detection

Peptides separated by the liquid chromatography were ionized through a nanoESI source and then entered a tandem mass spectrometer Orbitrap Exploris 480 (Thermo Fisher Scientific, San Jose, CA) for DDA (Data Dependent Acquisition) mode detection.

### DIA Quantitative Detection

Peptides separated by the liquid chromatography were ionized through a nanoESI source and then entered the tandem mass spectrometer Orbitrap Exploris 480 (Thermo Fisher Scientific, San Jose, CA) for DIA (Data Independent Acquisition) mode detection.

More specifically, in the instant study, the panel of genes evaluated included mitogen-activated protein kinase 5, identified in the scientific literature as MAP3K5, which is known to promote stress response and inhibit cell proliferation when expressed in cells; Intraflagellar transport protein 57, identified in the scientific literature as IFT57, which is known to promote cell apoptosis when expressed in cells; and Multifunctional ROCO Family Signaling Regulator 1, identified in the scientific literature as MFHAS1, which is known to promote inflammatory response when expressed in cells. Expression of any one or more of these genes is known to contribute to the deleterious effects on cells, as described above, and can directly and adversely impact the aging process of tissue, including skin.

As shown in the example herein, the claimed combination of skin actives including blueberry extract, pomegranate extract, dipotassium glycyrrhizate, and hydroxypropyl tetrahydropyrantriol demonstrated a statistically significant reduction in gene expression for each of MAP3K5, IFT57, and MFHAS1 which would thus confer reduction of cell stress response and cell proliferation, cell apoptosis, and inflammatory response in the tested cells.

Test compositions were prepared having the ingredients as shown in Table 2, where Table 2A shows the various test compositions and Table 2B shows the amounts of the tested ingredients and combinations.

TABLE 2A
Test Compositions

| COMBINATION | Composition |
|---|---|
| Proxylane + Blueberry + Pomegranate | A |
| Proxylane | B |
| Blueberry | C |
| Pomegranate | D |
| Dipotassium Glycyrrhizate | E |
| Proxylane + Blueberry + Pomegranate + | F, INVENTIVE |
| Dipotassium Glycyrrhizate | |

TABLE 2B
Test Compositions Formulations

| | COMPOSITION TESTED | | | | | |
|---|---|---|---|---|---|---|
| Ingredient | A | B | C | D | E | F |
| Proxylane | 0.109375% | 0.109375% | | | | 0.109375% |
| Blueberry | 0.000833% | | 0.000833% | | | 0.000833% |
| Pomegranate | 0.000208% | | | 0.000208% | | 0.000208% |
| Dipotassium | | | | | 0.002083% | 0.002083% |
| Glycyrrhizate | | | | | | |

The proteomics test results are expressed in LOG 2FC, as shown in Table 3 below.

Log 2FC Definition: Log 2FC represents the logarithm (base 2) of the ratio of expression levels between two conditions (e.g., treated vs. control). Interpretation: generally, a positive number indicates that the expression of the evaluated gene has increased, while a negative number indicates that the expression of the evaluated protein has decreased. For example, Log 2FC=1 shows that expression of the evaluated gene is doubled in the treatment group; Log 2FC=−1 shows that expression is halved in the treatment group; and, Log 2FC=0 shows there is no change in expression.

In the instantly reported study, each of panel of proteins known to be biomarkers associated with aging were evaluated. A positive Log 2FC score indicates an increase in protein expression which would be expected to be correlated with increased damaging effects on cells and thus and corresponding negative impact on skin aging, whereas, a negative Log 2FC score indicates a decrease in gene expression which would be expected to be correlated with conditions that diminish the damaging effects on cells and thus diminish the effects that manifest as skin aging.

As reflected in the data summarized below in Table 3, the inventors unexpectedly found that the claimed combination of skin actives according to the disclosure comprising blueberry extract, pomegranate extract, dipotassium glycyrrhizate, and hydroxypropyl tetrahydropyrantriol provides a statistically significant reduction in the expression of the genes, the reduction in the range from about 40% for MAP3K5 and about 50% for each of IFT57 and MFHAS1. Unexpectedly, this effect on gene expression is significantly greater in the claimed combination than a combination of only blueberry extract, pomegranate extract, and hydroxypropyl tetrahydropyrantriol.

TABLE 3
Proteomics Study Results

Protein Expression Evaluated

| | MAP3K5 | IFT57 | MFHAS1 |
|---|---|---|---|
| COMBINATION | Log2FC | Log2FC | Log2FC |
| Proxylane + Blueberry | −0.423 | 0.056* | −0.673 |
| extract + Pomegranate | | | |
| extract | | | |
| Proxylane | −0.265 | 0.171* | 0.051* |
| Blueberry extract | −0.268 | −0.608 | 0.904* |
| Pomegranate extract | −0.629 | −0.715 | −0.379 |
| Dipotassium | −0.298 | −1.035** | 0.543* |
| Glycyrrhizate | | | |
| Proxylane + Blueberry | −0.786** | −1.021** | −0.947** |
| extract + Pomegranate | | | |
| extract + Dipotassium | | | |
| Glycyrrhizate | | | |

*reflects an increase in gene expression
**Statistically Significant (P-Value, <0.05)

### Example 3: Exemplary Inventive Compositions

A representative example of an inventive composition comprising the claimed combination of skin actives comprising blueberry extract, pomegranate extract, dipotassium glycyrrhizate, and hydroxypropyl tetrahydropyrantriol is shown in Table 4.

Table 4A provides a representative embodiment according to the invention comprising the blend of claimed skin actives in a generic carrier system that may be, without being limiting, selected from a serum, oil in water or water in oil emulsion, a silicon emulsion thick cream, or some other form.

TABLE 4A
Inventive 1 (Amount in the table below reflects final wt % of active
in composition for those actives that are less than 100% in the RM)

| | INVENTIVE 1 | Range |
|---|---|---|
| INGREDIENT | (wt %) | (wt %) |
| Dipotassium Glycyrrhizate | 0.20 | ~0.05-5 |
| Hydroxypropyl Tetrahydropyrantriol | 10.5 | ~5-15 |
| Pomegranate Extract (Punica Granatum Fruit Extract) | 0.02 | ~0.01-5 |
| Blueberry Extract (Vaccinium Myrtillus Fruit Extract) | 0.08 | ~0.01-5 |
| Carrier; Water, Oils, Surfactant/Emulsifiers, Hydrating | 0.543 | QS |
| Agents, Additives | | |

Table 4B provides a representative embodiment according to the invention comprising the blend of claimed skin actives in a carrier system in the form of a silicon emulsion thick cream.

TABLE 4B
Inventive 2 (Amount in the table below reflects final wt % of active
in composition for those actives that are less than 100% in the RM)

| | INVENTIVE 2 Range |
|---|---|
| INGREDIENT | (wt %) |
| Dipotassium Glycyrrhizate | ~0.05-0.5 |
| Hydroxypropyl Tetrahydropyrantriol | ~5-15 |
| Pomegranate Extract (Punica Granatum Fruit Extract) | ~0.01-0.1 |
| Blueberry Extract (Vaccinium Myrtillus Fruit Extract) | ~0.01-0.5 |
| Adenosine, Tocopherol, Methyldihydrojasmonate | ~0.0005-2, each ingredient |
| Dimethicone | ~5-25 |
| Lauryl PEG-9 Polydimethylsiloxyethyl Dimethicone | ~0.5-15 |
| Dimethicone/PEG-10/15 Crosspolymer | ~0.5-15 |
| Synthetic Wax | ~0.5-10 |
| Ethylhexyl Hydroxystearate | ~0.05-2.5 |
| Isohexadecane | ~0.5-15 |
| Glycerin, Propylene Glycol, Dipropylene Glycol | ~0.05-20, each ingredient |
| Aluminum Starch Octenylsuccinate, Bismuth Oxychloride, | ~0-15, each ingredient |
| Cellulose, Silica Silylate | |
| Phenoxyethanol | ~0-5 |
| Sorbitol, Sodium Citrate, Magnesium Sulfate, Trisodium | ~0-2, each ingredient |
| Ethylenediamine Disuccinate | |
| Fragrance | ~0-2 |
| Water | QS |
| Total | 100 |

In some embodiments, cosmetic composition may include the following ingredients in amounts based on the total weight of the cosmetic composition that include: Dipotassium Glycyrrhizate (~0.20 wt %), Hydroxypropyl Tetrahydropyrantriol (~10.5 wt %), Punica granatum Fruit Extract (~0.02 wt %), Vaccinium myrtillus Fruit Extract (~0.08 wt %), a combination of Adenosine, Tocopherol, and Methyldihydrojasmonate (~0.543 wt %), a combination of Dimethicone, Lauryl PEG-9 Polydimethylsiloxyethyl Dimethicone, and Dimethicone/PEG-10/15 Crosspolymer (~20.7 wt %), a combination of Synthetic Wax, and Ethylhexyl Hydroxystearate (~4.3 wt %), Isohexadecane (~5.00 wt %), a combination of Glycerin, Propylene Glycol, and Dipropylene Glycol (~18.92 wt %), a combination of Aluminum Starch Octenylsuccinate, Bismuth Oxychloride, Cellulose, and Silica Silylate (~3.52 wt %), Phenoxyethanol (~0.70 wt %), a combination of Sorbitol, Sodium Citrate, Magnesium Sulfate, and Trisodium Ethylenediamine Disuccinate (~1.22 wt %), Fragrance (~0.10 wt %), and Water (QS, or ~36.55 wt %).

### Definitions and Terms

The transitional terms “comprising,” “consisting essentially of” and “consisting of,” when used in the appended claims, in original and amended form, define the claim scope with respect to what unrecited additional claim elements or steps, if any, are excluded from the scope of the claim(s). As used herein, the terms “comprising,” “having,” and “including” (or “comprise,” “have,” and “include”) are used in their open, non-limiting sense. The term “comprising” is intended to be inclusive or open-ended and does not exclude any additional, unrecited element, method, step, or material. The term “consisting of” excludes any element, step, or material other than those specified in the claim and, in the latter instance, impurities ordinary associated with the specified material(s). The term “consisting essentially of” limits the scope of a claim to the specified elements, steps, or material(s) and those that do not materially affect the basic and novel characteristic(s) of the claimed invention. All materials and methods described herein that embody the present invention can, in alternate embodiments, be more specifically defined by any of the transitional terms “comprising,” “consisting essentially of,” and “consisting of.”

In this application, the use of the singular includes the plural unless specifically stated otherwise. The singular forms “a,” “an,” “the,” and “at least one” are understood to encompass the plural as well as the singular unless the context clearly dictates otherwise, and these expressions, as well as the expression “one or more” which means “at least one,” are expressly intended to include the individual components as well as mixtures/combinations thereof.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. All ranges and amounts given herein are intended to include sub-ranges and amounts using any disclosed point as an end point.

A range given of “about 3% to 7%” is intended to have the term “about” modifying both the 3% and the 7% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

“Active material” as used herein with respect to the percent amount of an ingredient or raw material, refers to 100% activity of the ingredient in the raw material except as specifically stated. All amounts given herein are relative to the amount of active material, unless otherwise indicated. All percentages, parts and ratios herein are based upon the total weight of embodiments of the composition of the present disclosure, unless otherwise indicated.

“Skin care actives” are ingredients included in the formulation to provide specific benefits to the skin, such as exfoliation, brightening, and improvement of texture and tone.

“Fatty compounds” are lipophilic substances that provide moisturizing properties and enhance the emollience of the creamy brightening cleanser, contributing to skin softness and hydration.

“Additives” include a range of optional components such as preservatives, fragrances, and UV filters, which enhance the overall sensory experience and stability of the product.

“Surfactants” are surface-active agents that lower the surface tension between two substances, such as oil and water, aiding in the formation of stable emulsions and effective cleansing. As used herein, the term “surfactants,” as well as any specifically identified surfactants, includes salts of the surfactants even if not explicitly stated.

“Thickener” refers to ingredients that increase the viscosity of the creamy brightening cleanser, providing a desirable texture and stability without affecting the active components' efficacy.

“Stability” of embodiments of the composition according to the disclosure is demonstrated by a texture that does not break (i.e., no release of droplets) and does not demonstrate phase separation, precipitation, cracking or thinning over storage at ambient temperatures. In particular, the inventors have demonstrated that in inventive compositions according to the disclosure including, for example, a combination of Dimethicone, Lauryl PEG-9 Polydimethylsiloxyethyl Dimethicone, and Dimethicone/PEG-10/15 Crosspolymer at ~20.7 wt %, provides a robust cream having an inverse emulsion that maintains stability at industrial scale conditions, including at temperatures at least up to 40° C., wherein stability is demonstrated by absence of fluid pooling during storage at temperatures at least up to 40° C. A representative example includes the combination of ingredients in amounts based on the total weight of the cosmetic composition that include: Dimethicone present at about 15.64% by weight, based on the total weight of the cosmetic composition, Lauryl PEG-9 Polydimethylsiloxyethyl Dimethicone present at about 1.12% by weight, based on the total weight of the cosmetic composition, and PEG-10/15 Crosspolymer present at about 3.75% by weight, based on the total weight of the cosmetic composition. Thus, inclusion of silicones and silicone emulsifiers representing about 20% or more of the total weight of the composition beneficially confers stability including at industrial scale.

As used herein, the term “synthetic” means a material that is not of natural origin. The term “natural” and “naturally-sourced” and “nature-based” means a material of natural origin, such as derived from plants, which also cannot be subsequently chemically or physically modified. “Plant-based” means that the material came from a plant.

Unless otherwise expressly stated, it is in no way intended that any method set forth herein be construed as requiring that its steps be performed in a specific order. Accordingly, where a method claim does not expressly recite an order to be followed by its steps or it is not specifically stated in the claims or descriptions that the steps are to be limited to a specific order, it is no way intended that any particular order be inferred.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements. The example that follows serves to illustrate embodiments of the present disclosure without, however, being limiting in nature.

While the invention has been described with reference to one or more embodiments, it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. A cosmetic composition comprising:
a. a blend of skin actives comprising blueberry extract; pomegranate extract; dipotassium glycyrrhizate; and hydroxypropyl tetrahydropyrantriol; and
b. a cosmetic carrier system,
wherein the cosmetic composition confers a statistically significant reduction in the expression of at least one gene selected from the group consisting of mitogen-activated protein kinase 5 [MAP3K5], Intraflagellar transport protein 57 [IFT57], Multifunctional ROCO Family Signaling Regulator 1 [MFHAS11, and combinations thereof.

2. The cosmetic composition according to claim 1, wherein the cosmetic carrier system comprises or more ingredients selected from the group consisting of water, oils, hydrating agents, thickeners, emulsifiers, surfactants, water based solvents, oil based solvents, additional skin actives, fillers, preservatives, chelating agents, pH adjusters, dispersants, pigments, effect pigments, dyes, colorants, powders, opacifiers, fragrances, and combinations thereof.

3. The cosmetic composition according to claim 1, wherein the cosmetic composition is an emulsion, a gel, a single phase, or a bi-phase.

4. The cosmetic composition according to claim 1, wherein the cosmetic composition is in the form of a gel-cream, cream, gel, lotion, toner, or serum, and is intended to leave on, rinse off, or use as a cleanser.

5. The cosmetic composition according to claim 1, wherein blueberry extract in the form of Vaccinium myrtillus fruit extract is present in a range in a range from about 0.01% to about 5%, by weight, based on the weight of the composition.

6. The cosmetic composition according to claim 1, wherein pomegranate extract in the form of Punica granatum fruit extract is present in a range in a range from about 0.01% to about 5%, by weight, based on the weight of the composition.

7. The cosmetic composition according to claim 1, wherein dipotassium glycyrrhizate is present in a range in a range from about 0.05% to about 5%, by weight, based on the weight of the composition.

8. The cosmetic composition according to claim 1, wherein hydroxypropyl tetrahydropyrantriol is present in a range in a range from about 0.5% to about 15%, by weight, based on the weight of the composition.

9. The cosmetic composition according to claim 1, wherein blueberry extract in the form of Vaccinium myrtillus fruit extract is present at about 0.08%, pomegranate extract in the form of Punica granatum fruit extract is present at about 0.02%, dipotassium glycyrrhizate is present at about 0.20%, and hydroxypropyl tetrahydropyrantriol is present at about 10.5%, all amounts by weight, based on the weight of the composition.

10. A cosmetic composition comprising:
a. a blend of skin actives comprising blueberry extract; pomegranate extract;
dipotassium glycyrrhizate; and hydroxypropyl tetrahydropyrantriol; and
b. a cosmetic carrier system comprising:
i. water;
ii. at least one silicone-based emulsifier;
iii. at least one silicone oil;
iv. at least one branched, liquid alkane; and
v. at least one hydrating agent,
wherein the cosmetic composition confers a statistically significant reduction in the expression of at least one gene selected from the group consisting of mitogen-activated protein kinase 5 [MAP3K5], Intraflagellar transport protein 57 [IFT57], Multifunctional ROCO Family Signaling Regulator 1 [MFHAS11, and combinations thereof.

11. The cosmetic composition according to claim 10, wherein the cosmetic carrier system comprises or more ingredients selected from the group consisting of additional skin actives, fillers, preservatives, chelating agents, pH adjusters, dispersants, pigments, effect pigments, dyes, colorants, powders, opacifiers, fragrances, and combinations thereof.

12. The cosmetic composition according to claim 10, wherein, in the cosmetic carrier system:
a. the at least one silicone-based emulsifier is selected from the group consisting of dimethicone/PEG-10/15 crosspolymer, lauryl PEG-9 polydimethylsiloxyethyl dimethicone, dimethicone/polyglycerin-3 crosspolymer, cetyl PEG/PPG-10/1 dimethicone, PEG-18/PPG-18 dimethicone, PEG-10 dimethicone, PEG/PPG-18/18 methicone, lauryl PEG/PPG-18/18 methicone, PEG-9 polydimethylsiloxyethyl dimethicone, and combinations thereof;
b. the at least one silicone oil is dimethicone;
c. the at least one branched, liquid alkane is selected from the group consisting of isohexadecane, isododecane, isodecane, C15-19 alkane, and combinations thereof;
d. the at least one hydrating agent is selected from the group consisting of glycerin, propylene glycol, dipropylene glycol, ethylhexyl hydroxystearate, synthetic wax, and combinations thereof; and
e. comprises at least one additive selected from the group consisting of aluminum starch octenylsuccinate, bismuth oxychloride, cellulose, silica silylate, phenoxyethanol, sorbitol, sodium citrate, magnesium sulfate, and trisodium ethylenediamine disuccinate.

13. The cosmetic composition according to claim 10, wherein, in the cosmetic carrier system:
a. the water is present in a range from about 30% to about 40%;
b. the at least one silicone-based emulsifier comprises dimethicone/PEG-10/15 crosspolymer, alone or in combination with lauryl PEG-9 polydimethylsiloxyethyl dimethicone, the dimethicone/PEG-10/15 crosspolymer present in a range in a range from about 3% to about 4%, the lauryl PEG-9 polydimethylsiloxyethyl dimethicone present in a range in a range from about 1% to about 1.5%;
c. the at least one silicone oil comprises dimethicone present in a range in a range from about 5% to about 25%;
d. the at least one branched, liquid alkane comprises isohexadecane present in a range from about 1% to about 15%;
e. the at least one hydrating agent comprises glycerin, propylene glycol, ethylhexyl hydroxystearate, and synthetic wax; and
f. at least one additive comprises aluminum starch octenylsuccinate, bismuth oxychloride, cellulose, silica silylate, phenoxyethanol, sorbitol, sodium citrate, magnesium sulfate, and trisodium ethylenediamine disuccinate all amounts by weight, based on the weight of the composition.

14. The cosmetic composition according to claim 13, wherein blueberry extract in the form of Vaccinium myrtillus fruit extract is present in a range in a range from about 0.01% to about 5%, pomegranate extract in the form of Punica granatum fruit extract is present in a range in a range from about 0.01% to about 5%, dipotassium glycyrrhizate is present in a range in a range from about 0.05% to about 5%, and hydroxypropyl tetrahydropyrantriol is present in a range in a range from about 0.5% to about 15%, all amounts by weight, based on the weight of the composition.

15. The cosmetic composition according to claim 13, wherein blueberry extract in the form of Vaccinium myrtillus fruit extract is present at about 0.08%, pomegranate extract in the form of Punica granatum fruit extract is present at about 0.02%, dipotassium glycyrrhizate is present at about 0.20%, hydroxypropyl tetrahydropyrantriol is present at about 10.5%, all amounts by weight, based on the weight of the composition.

16. A cosmetic composition comprising a gel-cream emulsion comprising:
a. a blend of skin actives comprising blueberry extract; pomegranate extract; dipotassium glycyrrhizate; and hydroxypropyl tetrahydropyrantriol, wherein blueberry extract in the form of Vaccinium myrtillus fruit extract is present in a range in a range from about 0.01% to about 5%, pomegranate extract in the form of Punica granatum fruit extract is present in a range in a range from about 0.01% to about 5%, dipotassium glycyrrhizate is present in a range in a range from about 0.05% to about 5%, and hydroxypropyl tetrahydropyrantriol is present in a range in a range from about 5% to about 15%; and
b. a cosmetic carrier system comprising:
i. water present in a range from about 30% to about 40%;
ii. at least one silicone-based emulsifier comprising dimethicone/PEG-10/15 crosspolymer and lauryl PEG-9 polydimethylsiloxyethyl dimethicone, the dimethicone/PEG-10/15 crosspolymer present in a range in a range from about 3% to about 4%, the lauryl PEG-9 polydimethylsiloxyethyl dimethicone present in a range in a range from about 1% to about 1.5%;
iii. at least one silicone oil comprising dimethicone present in a range in a range from about 5% to about 25%;
iv. at least one branched, liquid alkane comprising isohexadecane present in a range in a range from about 1% to about 15%;
v. at least one hydrating agent present in a range from about 5% to about 30% and comprising glycerin, propylene glycol, ethylhexyl hydroxystearate, and synthetic wax; and
vi. at least one additive comprising aluminum starch octenylsuccinate, bismuth oxychloride, cellulose, silica silylate, phenoxyethanol, sorbitol, sodium citrate, magnesium sulfate, and trisodium ethylenediamine disuccinate,
all amounts by weight, based on the weight of the composition,
wherein the cosmetic composition confers a statistically significant reduction in the expression of at least one gene selected from the group consisting of mitogen-activated protein kinase 5 [MAP3K5], Intraflagellar transport protein 57 [IFT57], Multifunctional ROCO Family Signaling Regulator 1 [MFHAS11, and combinations thereof, and wherein the cosmetic composition is intended to leave on, rinse off, or use as a cleanser.

17. The cosmetic composition according to claim 15, wherein blueberry extract in the form of Vaccinium myrtillus fruit extract is present at about 0.08%, pomegranate extract in the form of Punica granatum fruit extract is present at about 0.02%, dipotassium glycyrrhizate is present at about 0.20%, and hydroxypropyl tetrahydropyrantriol is present at about 10.5%, all amounts by weight, based on the weight of the composition.

18. The cosmetic composition according to claim 15, wherein:
a. propylene glycol is present at about 12.54%, by weight, glycerin is present at about 4.4%, synthetic wax is present at about 4.00%, ethylhexyl hydroxystearate is present at about 0.3%; and
b. the at least one additive comprises aluminum starch octenylsuccinate present at about 2.50%, bismuth oxychloride present at about 0.7%, cellulose present at about 0.10%, silica silylate present at about 0.20%, phenoxyethanol present at about 0.70%, sorbitol present at about 0.08%, sodium citrate present at about 0.03%, magnesium sulfate present at about 0.70%, and trisodium ethylenediamine disuccinate present at about 0.11%,
all amounts by weight, based on the weight of the composition.
